(19) United States
(12) Patent Application Publication
(10) Pub. No.: US 2026/0090915 A1
(43) Pub. Date: Apr. 2, 2026

(54) OPHTHALMIC LASER TREATMENT APPARATUS

(71) Applicant: NIDEK CO.,LTD., Gamagori-shi (JP)

(72) Inventors: Kohei MASUNAGA, Gamagori-shi (JP); Hiroki KONDO, Gamagori-shi (JP); Shigeyoshi NAKAI, Gamagori-shi (JP); Teruki TSUKAMOTO, Gamagori-shi (JP)

(21) Appl. No.: 19/339,333

(22) Filed: Sep. 25, 2025

(30) Foreign Application Priority Data
Sep. 30, 2024 (JP) 2024-171798

(51) Int. Cl. A61F 9/008

(52) U.S. Cl. CPC A61F 9/008; A61F 2009/00844; A61F 2009/00878

(57) ABSTRACT

An ophthalmic laser treatment apparatus irradiates therapeutic laser light to tissue of a patient's eye each time an execution instruction for irradiating the therapeutic laser light is input. The ophthalmic laser treatment apparatus includes: a laser irradiation optical system irradiating the therapeutic laser light to the patient's eye; an observation optical system causing an operator to observe an observation image of the patient's eye through an eyepiece lens; an internal display unit provided in the observation optical system and configured to display an image to the operator through the eyepiece lens; and a control unit.

### CROSS REFERENCE TO RELATED APPLICATION

This application is based on, and claims the benefit of priority from Japanese Patent Application No. 2024-171798 on Sep. 30, 2024. The entire disclosure of the above application is incorporated herein by reference.

### TECHNICAL FIELD

The present disclosure relates to an ophthalmic laser treatment apparatus, a method implemented by the ophthalmic laser treatment apparatus, and a storage medium storing computer program code for the ophthalmic laser treatment apparatus.

### BACKGROUND ART

Conventionally, in the ophthalmic field, when treating the tissue of a patient's eye with therapeutic laser light, a contact lens having a reflective surface is sometimes used. For example, a contact lens having a reflective surface may be used for treating the trabecular meshwork or the like existing in the anterior chamber angle of the eyeball.

Here, when performing treatment using therapeutic laser light with a contact lens having a reflective surface, a mechanism to assist the treatment has been proposed (for example, an ophthalmic laser treatment apparatus described in Patent Document 1 (JP2024-049342A)). In this document, when irradiating a patient's eye with therapeutic laser light using a contact lens, an aiming guide for assisting in adjusting the aiming position of the therapeutic laser light to an appropriate position is displayed to the operator via an internal display unit of the apparatus. The operator observes the aiming guide together with the tissue reflected on the reflective surface and adjusts the aiming position of the therapeutic laser light according to the observation result.

### SUMMARY

Meanwhile, as a contact lens, there exists one having a plurality of reflective surfaces. The inventors have studied a technique for appropriately assisting treatment with therapeutic laser light when performing treatment using a contact lens having a plurality of reflective surfaces.

A typical objective of the present disclosure is to provide an ophthalmic laser treatment apparatus capable of appropriately assisting treatment with therapeutic laser light when a contact lens having a plurality of reflective surfaces is used.

In a first aspect of the present disclosure, an ophthalmic laser treatment apparatus irradiates therapeutic laser light to tissue of a patient's eye each time an execution instruction for irradiating the therapeutic laser light is input. The ophthalmic laser treatment apparatus includes: a laser irradiation optical system irradiating the therapeutic laser light to the patient's eye; an observation optical system causing an operator to observe an observation image of the patient's eye through an eyepiece lens; an internal display unit provided in the observation optical system and configured to display an image to the operator through the eyepiece lens; and a control unit. A scheduled irradiation region is defined as a region including a plurality of irradiation spots to which the therapeutic laser light is scheduled to be irradiated while using a contact lens having a plurality of reflective surfaces that reflect the therapeutic laser light in a direction intersecting with an optical axis. The scheduled irradiation region is divided into a plurality of irradiation sections. The control unit is configured to execute: an irradiation plan acquisition step of acquiring an irradiation plan that defines, in accordance with an arrangement of the plurality of reflective surfaces, an irradiation order of the therapeutic laser light for the plurality of irradiation sections; and an aiming guide display step of controlling, in accordance with progress of the irradiation plan, the internal display unit to display an aiming guide for assisting the operator in adjusting an aiming position of the therapeutic laser light to an appropriate position.

In a second aspect of the present disclosure, a method is executed by a control unit of an ophthalmic laser treatment apparatus that irradiates therapeutic laser light to tissue of a patient's eye each time an execution instruction for irradiating the therapeutic laser light is input. The method includes: irradiating, with a laser irradiation optical system, the therapeutic laser light to the patient's eye; causing, with an observation optical system, an operator to observe an observation image of the patient's eye through an eyepiece lens; and displaying, with an internal display unit provided in the observation optical system, an image to the operator through the eyepiece lens. A scheduled irradiation region is defined as a region including a plurality of irradiation spots to which the therapeutic laser light is scheduled to be irradiated while using a contact lens having a plurality of reflective surfaces that reflect the therapeutic laser light in a direction intersecting with an optical axis. The scheduled irradiation region is divided into a plurality of irradiation sections. The method further comprises, with the control unit: acquiring an irradiation plan that defines, in accordance with an arrangement of the plurality of reflective surfaces, an irradiation order of the therapeutic laser light for the plurality of irradiation sections; and controlling, in accordance with progress of the irradiation plan, the internal display unit to display an aiming guide for assisting the operator in adjusting an aiming position of the therapeutic laser light to an appropriate position.

In a third aspect of the present disclosure, a non-transitory, computer readable, storage medium stores computer program code executed by a control unit of an ophthalmic laser treatment apparatus that irradiates therapeutic laser light to tissue of a patient's eye each time an execution instruction for irradiating the therapeutic laser light is input. The computer program code, when executed by the control unit, causes the ophthalmic laser treatment apparatus to perform: irradiating, with a laser irradiation optical system, the therapeutic laser light to the patient's eye; causing, with an observation optical system, an operator to observe an observation image of the patient's eye through an eyepiece lens; and displaying, with an internal display unit provided in the observation optical system, an image to the operator through the eyepiece lens. A scheduled irradiation region is defined as a region including a plurality of irradiation spots to which the therapeutic laser light is scheduled to be irradiated while using a contact lens having a plurality of reflective surfaces that reflect the therapeutic laser light in a direction intersecting with an optical axis. The scheduled irradiation region is divided into a plurality of irradiation sections. The computer program code further causes the ophthalmic laser treatment apparatus to perform: acquiring, with the control unit, an irradiation plan that defines, in accordance with an arrangement of the plurality of reflective surfaces, an irradiation order of the therapeutic laser light for the plurality of irradiation sections; and controlling, with the control unit in accordance with progress of the irradiation plan, the internal display unit to display an aiming guide for assisting the operator in adjusting an aiming position of the therapeutic laser light to an appropriate position.

According to the ophthalmic laser treatment apparatus of the present disclosure, it is possible to appropriately assist treatment with therapeutic laser light when a contact lens having a plurality of reflective surfaces is used.

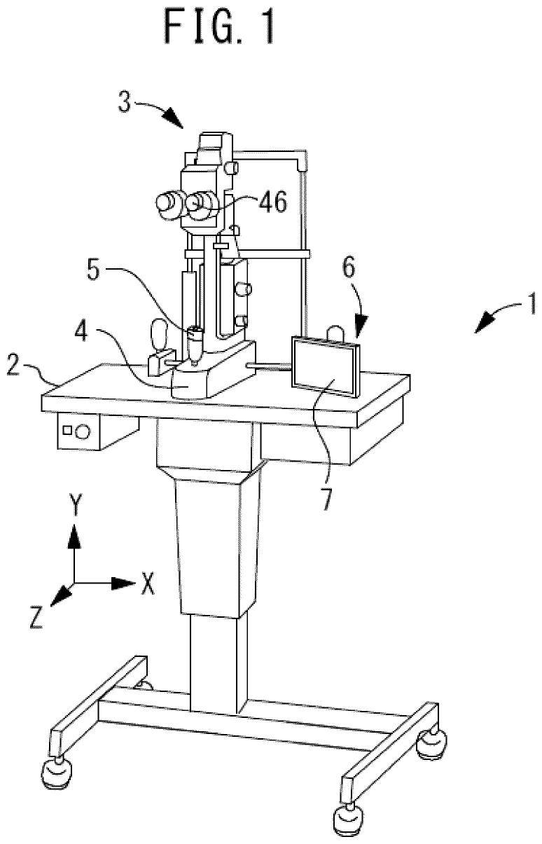

FIG. 1 is an external view of an ophthalmic laser treatment apparatus 1.

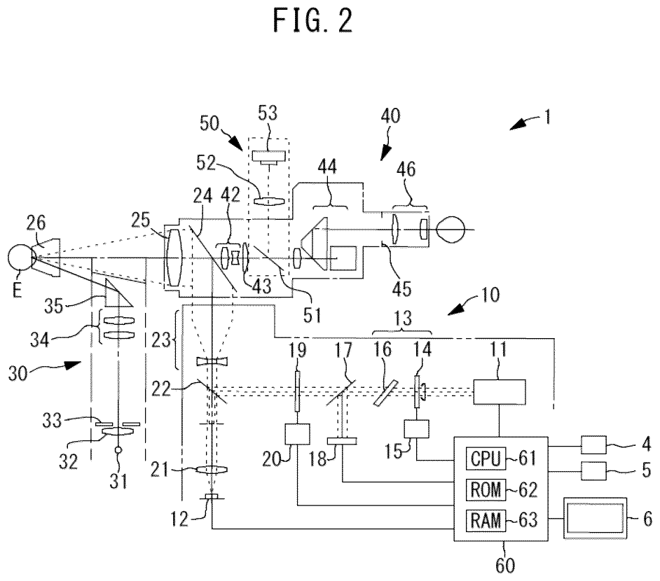

FIG. 2 is a view of an optical system of the ophthalmic laser treatment apparatus 1 as seen from a side of the system.

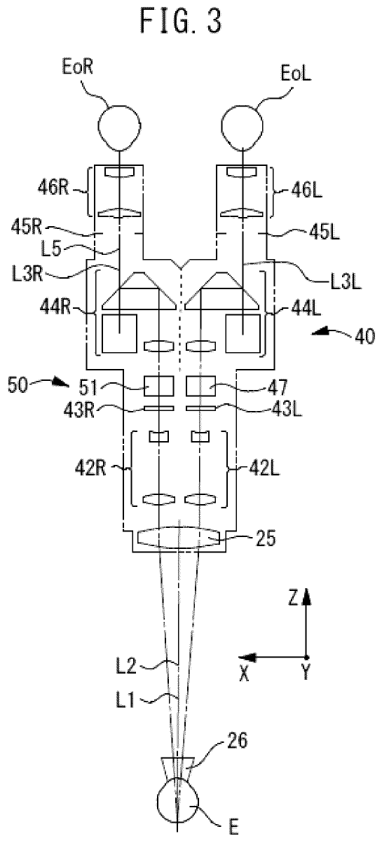

FIG. 3 is a view of the optical system of the ophthalmic laser treatment apparatus 1 as seen from an upper side of the system.

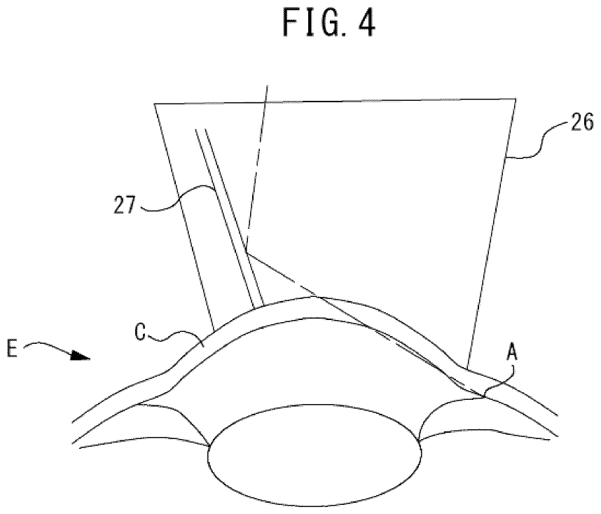

FIG. 4 is a schematic cross-sectional view of a patient's eye E and a contact lens 26.

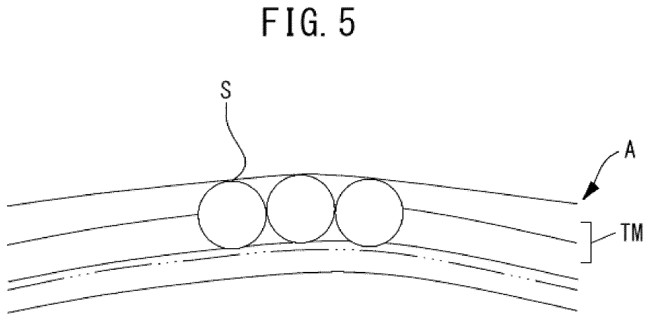

FIG. 5 is a schematic diagram illustrating an example of an observation region observed through a reflective surface 27 of the contact lens 26.

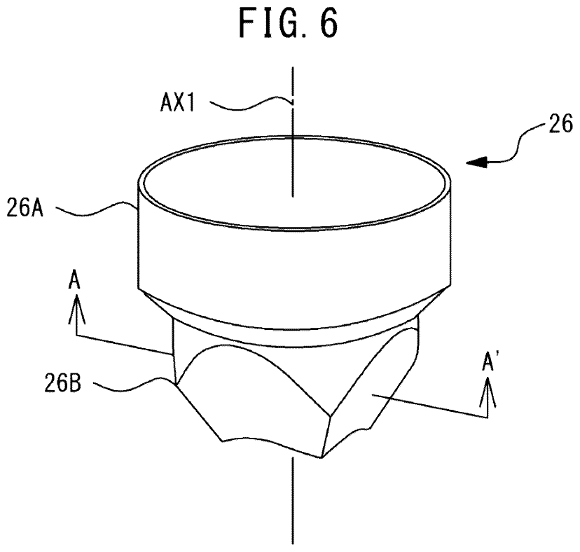

FIG. 6 is a perspective view of the contact lens 26.

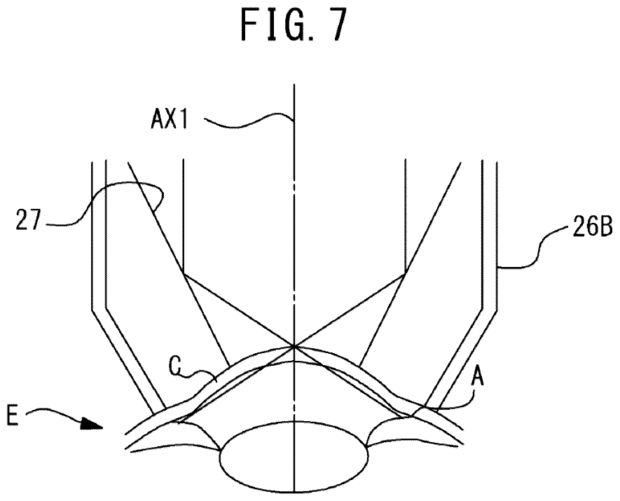

FIG. 7 is a view showing a cross-section of the contact lens 26 along line A-A′ illustrated in FIG. 6.

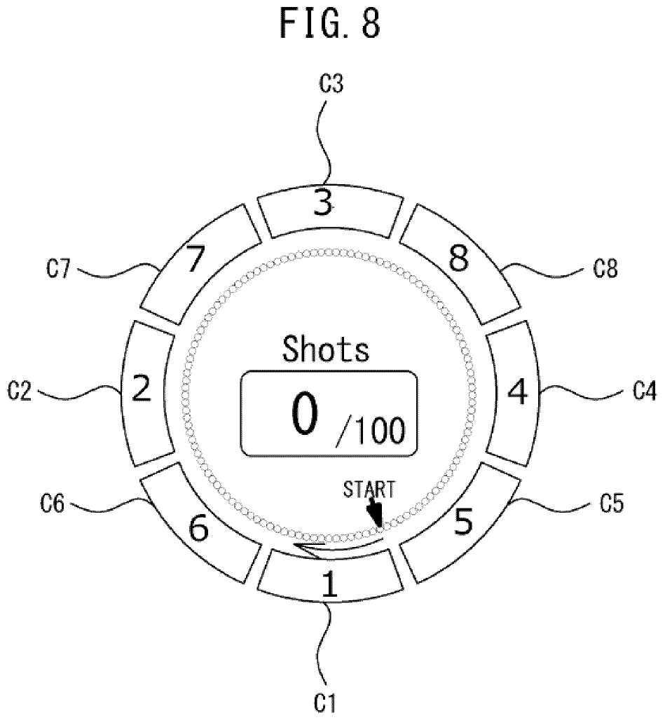

FIG. 8 is a diagram showing an example of an irradiation plan displayed on a control box 6.

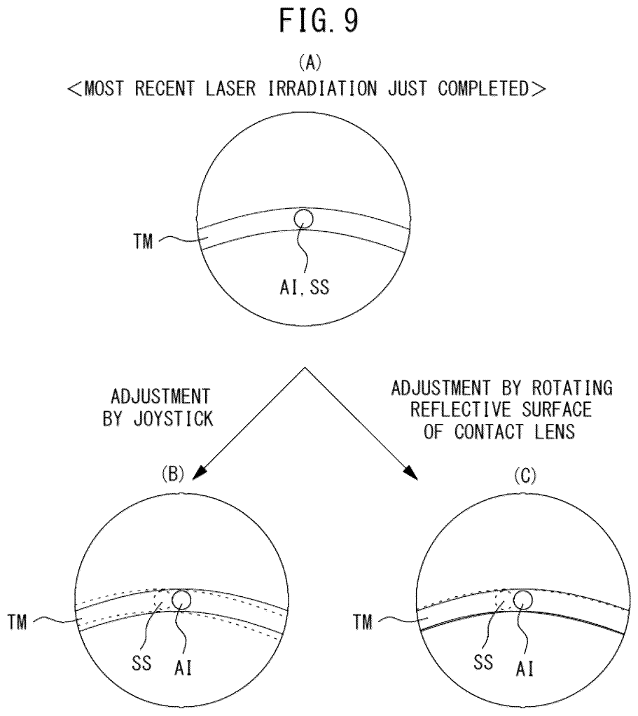

FIG. 9 is an explanatory diagram for explaining a method of adjusting an aiming position of an irradiation spot.

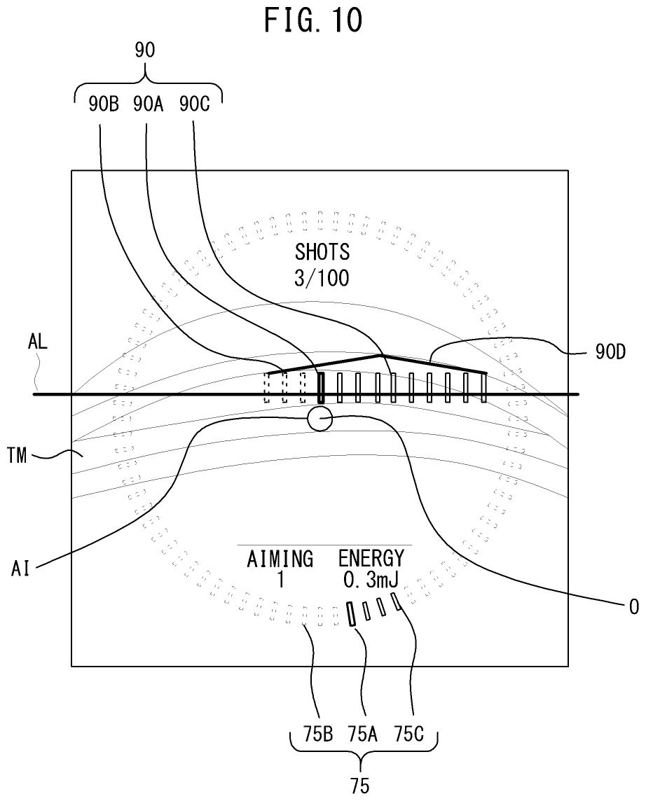

FIG. 10 is a diagram showing an example of an operator's observation field during treatment using the ophthalmic laser treatment apparatus 1 according to the embodiment.

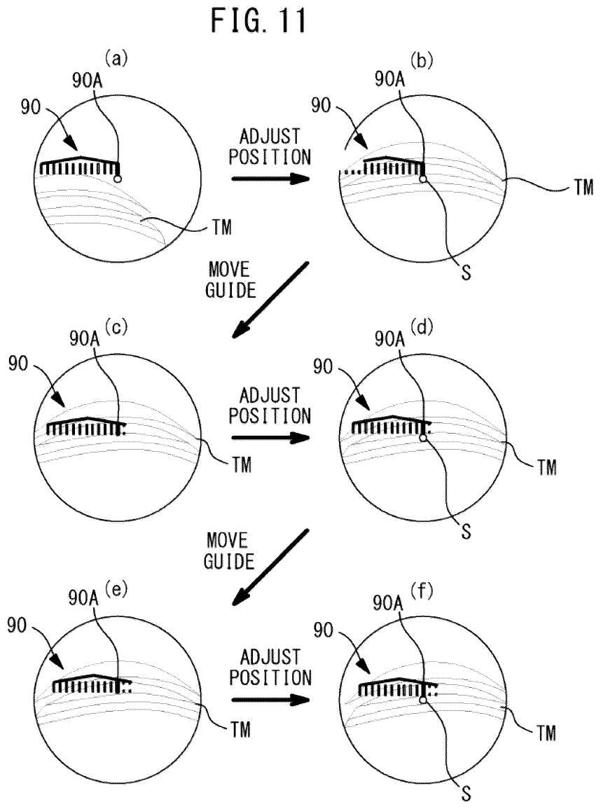

FIG. 11 is a diagram showing an example of a transition of a part of the operator's observation field during treatment using the ophthalmic laser treatment apparatus 1 according to the embodiment.

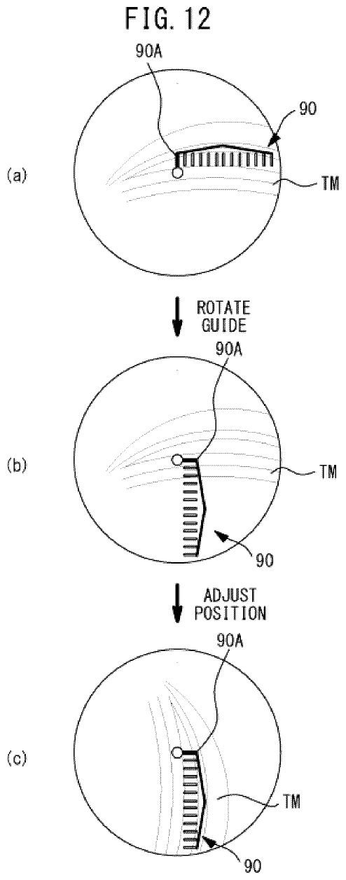

FIG. 12 is a diagram showing another example of a transition of a part of the operator's observation field during treatment using the ophthalmic laser treatment apparatus 1 according to the embodiment.

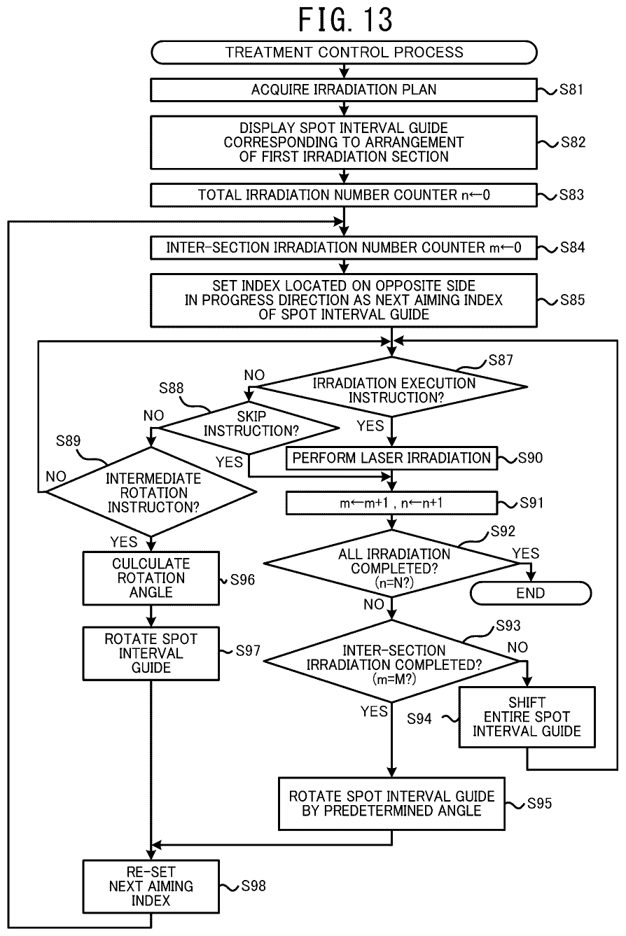

FIG. 13 is a flowchart of treatment control processing executed by the ophthalmic laser treatment apparatus 1 according to the embodiment.

### OVERVIEW

The ophthalmic laser treatment apparatus exemplified in the present disclosure irradiates therapeutic laser light to tissue of a patient's eye each time an execution instruction for irradiating the therapeutic laser light is input. The ophthalmic laser treatment apparatus includes a laser irradiation optical system, an observation optical system, an internal display unit, and a control unit. The laser irradiation optical system irradiates therapeutic laser light to the patient's eye. The observation optical system causes an operator to observe an observation image of the patient's eye through an eyepiece lens. The internal display unit is provided in the observation optical system and displays an image to the operator through the eyepiece lens. The control unit controls various operations of the ophthalmic laser treatment apparatus. The control unit executes an irradiation plan acquisition step and an aiming guide display step. A scheduled irradiation region is defined as a region including a plurality of irradiation spots to which the therapeutic laser light is scheduled to be irradiated while using a contact lens having a plurality of reflective surfaces that reflect the therapeutic laser light in a direction intersecting with an optical axis. The scheduled irradiation region is divided into a plurality of irradiation sections. In the irradiation plan acquisition step, the control unit acquires an irradiation plan that defines an irradiation order of therapeutic laser light to the plurality of irradiation sections according to an arrangement of the plurality of reflective surfaces.

In the contact lens, the plurality of reflective surfaces may have mutually different rotation angles with respect to a central axis. Further, two adjacent sections among the plurality of irradiation sections may partially overlap with each other or may be arranged without overlapping.

In the aiming guide display step, the control unit causes the internal display unit to display, according to progress of the irradiation plan, an aiming guide for assisting the operator in adjusting an aiming position of the therapeutic laser light to an appropriate position.

According to the ophthalmic laser treatment apparatus in the present disclosure, the aiming guide for assisting an operator in adjusting the aiming position is displayed by the internal display unit according to progress of the irradiation plan. Therefore, the operator can confirm the aiming guide while continuously observing the patient's eye through the eyepiece lens, that is, without moving the eye away from the eyepiece lens. Then, the operator can adjust the aiming position of the therapeutic laser light with reference to the confirmed aiming guide. Accordingly, the operator can adjust the aiming position of the therapeutic laser light more appropriately.

In this case, since the irradiation order of the therapeutic laser light for the plurality of irradiation sections is defined, as the irradiation plan, according to the arrangement of the plurality of reflective surfaces in the contact lens, the treatment with the therapeutic laser light when a contact lens having a plurality of reflective surfaces is used can be effectively assisted.

As the irradiation plan, at least the irradiation order of the therapeutic laser light for the plurality of irradiation sections may be defined according to the arrangement of the plurality of reflective surfaces in the contact lens. In this case, the plurality of irradiation sections in the scheduled irradiation region are broadly classified into first irradiation sections and second irradiation sections. Thereafter, the irradiation order of the therapeutic laser light for the plurality of irradiation sections is set such that, after the therapeutic laser light is irradiated onto all the first irradiation sections, the therapeutic laser light is irradiated onto each of the second irradiation sections. Here, the first irradiation sections are a plurality of irradiation sections each corresponding to a respective one of the plurality of reflective surfaces when the contact lens is fixed with the plurality of reflective surfaces at first angles. The second irradiation sections are a plurality of irradiation sections each corresponding to a respective one of the plurality of reflective surfaces when the contact lens is fixed with the plurality of reflective surfaces at second angles different from the first angles. That is, during irradiation of the therapeutic laser light onto each of the irradiation sections categorized as the first irradiation sections, the operator does not necessarily need to rotate the contact lens. Similarly, during irradiation of the therapeutic laser light onto each of the irradiation sections categorized as the second irradiation sections, the operator does not necessarily need to rotate the contact lens. Accordingly, operability for the operator can be improved.

A center of a display region in the internal display unit may coincide with an optical axis of the observation optical system (hereinafter also referred to as an “observation optical axis”). In this case, an image is displayed in the display region of the internal display unit with the observation optical axis of the observation optical system as a reference. Accordingly, the operator can appropriately perform treatment while recognizing the image presented at an appropriate position in the field of view.

An optical axis of the therapeutic laser light irradiated by the laser irradiation optical system may also coincide with the optical axis of the observation optical system. In this case, the control unit can display the image in an appropriate direction and at an appropriate angle centering on the optical axis of the therapeutic laser light. Further, when the laser irradiation optical system irradiates the patient's eye with aiming laser light (hereinafter also referred to as “aiming light”) for causing the operator to recognize an irradiation scheduled position of the therapeutic laser light, an optical axis of the aiming light may also coincide with the optical axis of the observation optical system. In this case, since the operator visually recognizes the aiming light at the center of the observation field and the display region of the internal display unit, adjustment of the irradiation position of the therapeutic laser light based on the aiming light and the display content becomes easier.

The control unit may control the internal display unit to display, as the aiming guide, a target site guide serving as a reference to which an arcuate or annular treatment target site of the patient's eye, observed through the observation optical system, is to be aligned. In this case, the operator can easily adjust the irradiation position of the therapeutic laser light to an appropriate position by making various adjustments so that the arcuate or annular treatment target site observed through the observation optical system matches the displayed target site guide.

The control unit may switch between displaying and not displaying, by the internal display unit, at least a part (for example, a straight portion or an arcuate portion) of the target site guide, according to an instruction input by the operator. In this case, when the therapeutic laser light is actually irradiated toward an irradiation spot, it is possible to prevent at least a part of the target site guide from obstructing the aiming of the therapeutic laser light.

The control unit may determine an angle of the target site guide to be displayed on the internal display unit according to progress of the irradiation plan, and may cause the target site guide to be displayed at the determined angle. In this case, since the target site guide is displayed on the internal display unit at an appropriate angle according to the progress of the irradiation plan, the aiming position can be appropriately adjusted.

For example, the control unit may determine an angle of the target site guide according to a position or a direction of the irradiation section defined by the irradiation plan. For example, each time irradiation of the therapeutic laser light with a predetermined number of shots scheduled for one irradiation section is completed, the control unit may rotate an angle of the target site guide by a specified rotation angle toward a direction corresponding to another irradiation section. In this case, when the irradiation section is changed among the first irradiation sections (or among the second irradiation sections), the target site guide is rotated by, as the specified rotation angle, a rotation angle substantially equal to an arrangement interval of the plurality of reflective surfaces in the contact lens. Further, when the irradiation section is changed between the first irradiation sections and the second irradiation sections, the target site guide is rotated by, as the specified rotation angle, a value corresponding to a difference between the first angle corresponding to the first irradiation sections and the second angle corresponding to the second irradiation sections, or a value obtained by adding an integer multiple of the arrangement interval of the plurality of reflective surfaces to the difference. Accordingly, when treatment is performed using the contact lens having the plurality of reflective surfaces, adjustment of the aiming position can be facilitated more appropriately.

The control unit may control the internal display unit to display, as the aiming guide, an angle guide indicating a rotation angle for the contact lens suitable for progress of the irradiation plan. In this case, the operator can refer to the angle indicated by the angle guide to adjust the rotation angle of the contact lens or to adjust a relative position of the apparatus with respect to the patient's eye. Accordingly, when treatment is performed using the contact lens having a plurality of reflective surfaces, it becomes easier to adjust the aiming position of the therapeutic laser light so that the reflective surfaces corresponding to progress of the irradiation plan are used.

The control unit may further execute a rotation recommendation step of recommending the operator to rotate the contact lens to the second angle when irradiation of the therapeutic laser light onto all the first irradiation sections has been completed. In this case, the operator can appropriately grasp the timing at which rotation of the contact lens is necessary.

A specific method for recommending rotation of the reflective surfaces of the contact lens can be selected as appropriate. For example, the control unit may recommend rotation of the reflective surfaces by at least one of sound and vibration. Further, the control unit may recommend rotation of the reflective surfaces by controlling display content of the internal display unit, for example, by rotating an image displayed on the internal display unit by a predetermined angle.

The control unit may cause the internal display unit to display, along a peripheral portion of an observation field observed by the operator through the observation optical system, a peripheral guide, as the aiming guide, which indicates at least one of a rotation angle of the contact lens suitable for progress of the irradiation plan and a direction of an irradiation spot to which the therapeutic laser light should be irradiated. In this case, the operator can easily recognize at least one of the appropriate rotation angle of the contact lens (the reflective surfaces) and the direction of the irradiation spot by visually recognizing the peripheral guide displayed along the peripheral portion of the observation field.

The peripheral guide may be displayed arcuately or annularly along the peripheral portion of the observation field. A center of the arcuate or annular peripheral guide may coincide with the optical axis of the observation optical system. In this case, a direction indicated by the peripheral guide coincides with a direction from a center of the observation field. Accordingly, the operator can appropriately recognize the direction indicated by the peripheral guide.

The peripheral guide may include a next aiming guide indicating a direction to a next irradiation spot among the plurality of irradiation spots defined by the irradiation plan. The control unit may move a position of the next aiming guide to a position corresponding to an adjacent irradiation spot in a progress direction defined by the irradiation plan each time irradiation of the therapeutic laser light is executed. In this case, the operator can appropriately recognize the direction of the next irradiation spot by the next aiming guide that is moved along the peripheral portion of the observation field.

The peripheral guide may include an irradiation completion guide indicating a direction to an irradiation completed spot among the plurality of irradiation spots defined by the irradiation plan. The control unit may change the next aiming guide displayed to the irradiation completion guide each time irradiation of the therapeutic laser light is executed. In this case, the operator can adjust the next aiming position appropriately with the next aiming guide while recognizing a direction in which irradiation of the therapeutic laser light has been completed. Further, the operator can also easily recognize progress of the treatment.

The peripheral guide may include an unirradiated guide indicating a direction to an irradiation spot, among the plurality of irradiation spots in the irradiation plan, that is scheduled to be irradiated with the therapeutic laser light after the next irradiation. The control unit may, each time irradiation of the therapeutic laser light is completed, change the unirradiated guide that has been displayed in a direction to the irradiation spot of the next irradiation order defined in the irradiation plan to the next aiming guide. In this case, while also recognizing a direction to an irradiation spot scheduled to be irradiated with the therapeutic laser light after the next irradiation, the operator can appropriately adjust the next aiming position with the next aiming guide. Further, the operator can easily grasp progress of the treatment.

The peripheral guide may include a section aiming guide that indicates either (i) a direction to an irradiation section, which is a section including a plurality of irradiation spots to which the therapeutic laser light is irradiated when rotation angles of the respective reflective surfaces of the contact lens are fixed or (ii) a direction to a rotation angle of the contact lens suitable for irradiation of the therapeutic laser light to the irradiation section. The control unit may, each time irradiation of the therapeutic laser light in the same number as the plurality of irradiation spots included in the irradiation section is completed, move the section aiming guide to a position adjacent in a progress direction defined by the irradiation plan. In this case, the operator can easily grasp, by the section aiming guide, either directions to the plurality of irradiation sections or a direction to the reflective surface for irradiating the therapeutic laser light onto the irradiation spots within the irradiation section. Accordingly, adjustment of the angle of the contact lens becomes easier.

The control unit may cause the section aiming guide to be displayed for each range of one specified angle in a partial rotation type lens. The control unit may move a position of the section aiming guide to an adjacent position in a progress direction defined by the irradiation plan each time irradiation of the therapeutic laser light the same number of times as a plurality of irradiation spots included in one specified angle range is completed. In this case, the section aiming guide is appropriately displayed according to specifications of the partial rotation type lens and progress of the treatment. Therefore, even when the partial rotation type lens is used, adjustment of the aiming position is appropriately assisted by the section aiming guide.

It is also possible to change a specific configuration of the aiming guide. For example, the control unit may control the internal display unit to display an image of a treatment target site that is irradiated with the aiming light and captured by the imaging unit based on irradiation of the previous therapeutic laser light. In this case, the operator can grasp the previous irradiation site of the therapeutic laser light (that is, the site where the aiming light appears in the displayed image), and can adjust the next aiming position based thereon. Accordingly, the therapeutic laser light can be more appropriately irradiated.

When at least one of the angle guide and the peripheral guide as described above is displayed as the aiming guide, even if the ophthalmic laser treatment apparatus displays the aiming guide on a display unit different from the internal display unit (for example, a display unit provided outside the observation optical system), adjustment of the aiming position by the operator can still be appropriately assisted.

### Embodiment

Hereinafter, a typical embodiment in the present disclosure will be described with reference to the drawings. The ophthalmic laser treatment apparatus 1 of the present embodiment is able to irradiate therapeutic laser light onto a patient's eye E to perform treatment of the patient's eye E.

### Overall Configuration

With reference to FIGS. 1 to 3, a configuration of the ophthalmic laser treatment apparatus 1 will be described. As shown in FIG. 1, the ophthalmic laser treatment apparatus 1 in the present embodiment includes a table unit 2, a main body unit 3, and a control box 6. The main body unit 3 and the control box 6 are installed on the table unit 2.

The main body unit 3 includes various components such as a laser irradiation optical system 10, an illumination optical system 30, an observation optical system 40, an internal display unit 50, and a control unit 60 (see FIG. 2). The main body unit 3 also includes a base unit 4 and a joystick unit 5 (operation lever). The base unit 4 is displacement means having a displacement mechanism. The base unit 4 moves at least one of the laser irradiation optical system 10, the observation optical system 40, and the internal display unit 50 in the vertical direction (Y direction in FIG. 1), the lateral direction (X direction in FIG. 1), and the longitudinal direction (Z direction in FIG. 1). By the displacement means, a positional relationship between the patient's eye and the laser irradiation optical system 10 is changed in the vertical direction, the lateral direction, and the longitudinal direction. The base unit 4 further rotates at least one of the laser irradiation optical system 10, the observation optical system 40, and the internal display unit 50 in a horizontal direction about an axis extending in the vertical direction as a rotation center. By operating the joystick unit 5, the operator can move or rotate the laser irradiation optical system 10, the observation optical system 40, and the internal display unit 50 to adjust an observation position of the patient's eye E and an irradiation position of the laser light (therapeutic laser light and aiming light). In this embodiment, the joystick unit 5 (for example, an upper end portion of the joystick 5) is provided with an operation button operated by the operator. In this embodiment, the operation button of the joystick unit 5 is used as a trigger input means for inputting a trigger to execute irradiation of the therapeutic laser light. It is also possible that a foot switch or the like operated by the operator's foot is used as the trigger input means for inputting a trigger to execute irradiation of the therapeutic laser light.

The control box 6 includes an external display unit 7 provided outside the observation optical system 40 (see FIG. 2). The external display unit 7 can display various images. A touch panel type operation unit is provided on a surface of the external display unit 7 of the control box 6. The control box 6 controls the external display unit 7 to display various parameters relating to treatment and also receives various instruction inputs from the user.

### Laser Irradiation Optical System

As shown in FIG. 2, the laser irradiation optical system 10 of the present embodiment includes a therapeutic laser light source 11, an aiming light source 12, an energy adjusting unit 13, a beam splitter 17, a photodetector 18, a safety shutter 19, a collimator lens 21, a dichroic mirror 22, an expander lens 23, a dichroic mirror 24, and an objective lens 25.

The therapeutic laser light source 11 emits therapeutic laser light for treating tissue of the patient's eye E. As one example, in the laser light source 11 of the present embodiment, a YAG (yttrium aluminum garnet) crystal doped with neodymium (Nd: YAG) is used as a laser rod. Further, a wavelength conversion element (not shown) can convert infrared laser light (wavelength: 1064 nm) emitted by the laser light source 11 into visible laser light (wavelength: 532 nm).

The aiming light source 12 emits aiming laser light (hereinafter also referred to simply as “aiming light”) that indicates a position to be irradiated with the therapeutic laser light (that is, a position of an irradiation spot). In the present embodiment, a light source that emits visible laser light having a wavelength of 635 nm (red) is used as the aiming light source 12. However, it goes without saying that the wavelength of the aiming light and the like can be appropriately changed.

The energy adjusting unit 13 adjusts an amount of energy of the therapeutic laser light irradiated onto tissue of the patient's eye E. The energy adjusting unit 13 of the present embodiment includes a half-wave plate 14 and a polarizing plate 16. The half-wave plate 14 rotates about the optical axis of the therapeutic laser light by a motor 15. The polarizing plate 16 is disposed at the Brewster angle. By a combination of the half-wave plate 14 and the polarizing plate 16, the amount of energy of the therapeutic laser light is adjusted.

The beam splitter 17 reflects a part of the therapeutic laser light toward the photodetector 18. The photodetector 18 detects the amount of energy of the therapeutic laser light by receiving the therapeutic laser light reflected by the beam splitter 17. The safety shutter 19 moves between an on-axis position and an off-axis position of the therapeutic laser light by a shutter driving unit (for example, a solenoid) 20. By being disposed on the optical axis of the therapeutic laser light, the safety shutter 19 blocks irradiation of the therapeutic laser light onto the patient's eye E.

The collimator lens 21 makes the aiming light emitted from the aiming light source 12 into a parallel light beam. The dichroic mirror 22 combines the therapeutic laser light and the aiming light coaxially. In the present embodiment, the dichroic mirror 22 reflects the therapeutic laser light and transmits the aiming light, thereby combining the therapeutic laser light and the aiming light.

The expander lens 23 enlarges a light flux of the laser light (the therapeutic laser light and the aiming light) combined by the dichroic mirror 22. The laser light enlarged by the expander lens 23 is reflected by the dichroic mirror 24 and passes through the objective lens 25. In the present embodiment, the laser light that has passed through the objective lens 25 is irradiated onto tissue of the patient's eye E via a contact lens 26 attached to the patient's eye E. The dichroic mirror 24 reflects light of a wavelength of the therapeutic laser light reflected by the patient's eye E so that the reflected therapeutic laser light hardly enters the operator's eye. The irradiation optical system 10 may further include a configuration for adjusting a spot size of the laser light irradiated onto tissue.

### Illumination Optical System

The illumination optical system 30 illuminates an observation site including a treatment target site of tissue. The illumination optical system 30 in the present embodiment includes a lamp 31, a lens 32, a diaphragm 33, a lens group 34, and a prism 35. For example, a white light emitting element may be used as the lamp 31. The illumination optical system 30 may also include a slit plate or the like for illuminating the observation site with slit light.

### Observation Optical System

As shown in FIGS. 2 and 3, the observation optical system 40 is an observation means that allows the operator to observe the patient's eye E and includes an optical axis L3 (see FIG. 3). As shown in FIG. 3, the observation optical system 40 of the present embodiment includes an optical axis L3R for presenting an observation image to the operator's right eye EoR, and an optical axis L3L for presenting an observation image to the operator's left eye EoL. The observation optical system 40 of the present embodiment may also be referred to as a binocular microscope. The observation optical system 40 in the present embodiment includes an objective lens 25, a variable magnification optical system 42 (42R, 42L), protective filters 43 (43R, 43L), a half mirror 47, an erect prism group 44 (44R, 44L), field diaphragms 45 (45R, 45L), and eyepiece lenses 46 (46R, 46L). By looking into the eyepiece lens 46, the operator can confirm an observation site of the patient's eye E, a spot of the aiming light (in other words, reflected light (returning light) of the aiming light reflected by the patient's eye E), and the like. In the present embodiment, an observation surface (object surface) provided at the tip of the objective lens 41 and the field diaphragm 45 disposed inside the apparatus are in an optically conjugate positional relationship via the objective lens 41. That is, at the position of the field diaphragm 45, an observation image of the patient's eye E is formed as a real image in space. In the present embodiment, the magnification of the observation image observed by the operator is changed by the variable magnification optical system 42. The observation optical system 40 is provided with an encoder (not shown) for acquiring the magnification of the observation image by the variable magnification optical system 42.

### Internal Display Unit

As shown in FIG. 2, the internal display unit 50 is provided in the observation optical system 40 and displays an image to the operator through the eyepiece lens 46. The internal display unit 50 includes a display 53, a lens 52, and a half mirror 51. Various images are displayed on the display 53. In the present embodiment, an LCD (with a backlight) is used as the display 53. Specifically, in the present embodiment, a color LCD capable of displaying 1600 (H)×1200 (V) is used as the display 53. As shown in FIG. 3, the half mirror 51 is disposed on the optical axis L3R. Specifically, the half mirror 51 is disposed between the protective filter 43R and the erect prism group 44R.

Presentation light (display light) emitted from the display 53 travels along the optical axis L5 (see FIG. 3). Specifically, the presentation light emitted from the display 53 passes through the lens 52 and is reflected by the half mirror 51 toward the erect prism group 44R. The half mirror 51 of the present embodiment is a combining means for combining an optical observation image observed by the observation optical system 40 with an image displayed on the display 53. The half mirror 51 of the present embodiment makes the optical axis L5 coaxial with the optical axis L3R. The presentation light reflected by the half mirror 51 travels through the erect prism group 44R, the field diaphragm 45R, and the eyepiece lens 46R in this order, and is focused on the fundus of the operator's eye looking into the eyepiece lens 46R. The internal display unit 50 functions as a so-called head-up display (HUD).

In the present embodiment, the display 53 and the field diaphragm 45R are in an optically conjugate positional relationship. That is, at the position of the field diaphragm 45R, a display image of the display 53 is formed as a real image in space. It should be noted that the method of displaying an image to the operator through the eyepiece lens 46 is not limited to the method exemplified in the present disclosure. For example, as the internal display unit, an LCD (liquid crystal panel) without a backlight may be disposed at the position of the field diaphragm 45R (on the optical axis L3R), and the control unit 60 may control transmittance of each cell constituting the LCD to present display information to the operator.

In the present embodiment, a center of a display region of the internal display unit 50 coincides with the observation optical axis of the observation optical system 40. Accordingly, with the observation optical axis of the observation optical system 40 as a reference, an image is displayed in the display region of the internal display unit 50. Therefore, the operator can appropriately perform treatment while recognizing the image presented at an appropriate position in the observation field through the eyepiece lens 46.

In the present embodiment, the optical axis of the therapeutic laser light irradiated by the laser irradiation optical system 10 also coincides with the observation optical axis of the observation optical system 40 and with the center of the display region of the internal display unit 50. Accordingly, the internal display unit 50 can display an image in an appropriate direction and at an appropriate angle centering on the optical axis of the therapeutic laser light. Furthermore, in the present embodiment, the optical axis of the aiming light irradiated by the laser irradiation optical system 10 also coincides with the observation optical axis of the observation optical system 40 and with the center of the display region of the internal display unit 50. Therefore, since the operator visually recognizes the aiming light at the center of the observation field and at the center of the display region of the internal display unit 50, adjustment of the irradiation position of the therapeutic laser light based on the aiming light and the display content becomes easier.

Although illustration is omitted, the observation optical system 40 of the present embodiment is provided with an imaging optical system for capturing an observation image of the patient's eye E and the like. The imaging optical system includes a half mirror, an imaging lens, and an imaging element. The half mirror is disposed in one of the left or right observation optical paths provided in the observation optical system 40. Light incident from the observation site and the like through the objective lens 25 onto the half mirror is reflected by the half mirror and enters the imaging element through the imaging lens. As a result, the imaging optical system captures the observation image.

### Control Unit

The control unit 60 controls various operations of the laser treatment apparatus 1. The control unit 60 of the present embodiment includes a CPU (processor) 61, a ROM 62, a RAM 63, and a nonvolatile memory 65. The CPU 61 controls respective components of the laser treatment apparatus 1. The ROM 62 stores various programs, initial values, and the like. The RAM 63 temporarily stores various information. The nonvolatile memory 65 is a non-transitory storage medium that can retain stored contents even if supply of electric power is interrupted. For example, a USB memory removably attached to the control unit 60 or a flash ROM incorporated in the control unit 60 can be used as the nonvolatile memory 65. In the present embodiment, the control unit 60 is connected to the base unit 4, the joystick unit 5, the control box 6, the therapeutic laser light source 11, the aiming light source 12, the motor 15, the photodetector 18, the shutter driving unit 20, the lamp 31, and the display 53.

In the present disclosure, the term “processor” may refer to a single hardware processor or several hardware processors that are configured to execute computer program code (i.e., one or more instructions of a program) included in a program. In other words, a processor may be one or more programmable hardware devices. For instance, a processor may be a general-purpose or embedded processor and include, but not necessarily limited to, CPU (a Central Processing Circuit), a microprocessor, GPU (a Graphics Processing Unit), and DFP (a Data Flow Processor).

The term “memory” in the present disclosure is a non-transitory, tangible storage medium, and may refer to a single or several hardware memory configured to store computer program code (i.e., one or more instructions of a program) and/or data accessible by a processor. A memory may be implemented using any suitable memory technology, such as static random-access memory (SRAM), synchronous dynamic RAM (SDRAM), nonvolatile/Flash-type memory, or any other type of memory. Computer program code constituting a program may be stored on the memory and, when executed by a processor, cause the processor to perform various functions which will be described below.

In the present disclosure, the term “circuit” may refer to a single hardware logical circuit or several hardware logical circuits (in other words, “circuitry”) that are configured to cause the ophthalmic laser treatment apparatus 1 to perform one or more functions. In other words (and in contrast to the term “processor”), the term “circuit” refers to one or more non-programmable devices. For instance, a circuit may be a customized IC (an Integrated Circuit) that is customized for a particular use and is configured as non-programmable.

In the present disclosure, the phrase “at least one of (i) a circuit and (ii) a processor” should be understood as disjunctive (logical disjunction) where the circuit and the processor can be optional and not be construed to mean “at least one of a circuit and at least one of a processor”. Therefore, in the present disclosure, the phrase “at least one of a circuit and a processor is configured to cause the ophthalmic laser treatment apparatus 1 to perform functions” should be understood as “only the circuit can cause the ophthalmic laser treatment apparatus 1 to perform all the functions”. Further, the phrase “at least one of a circuit and a processor is configured to cause the ophthalmic laser treatment apparatus 1 to perform functions” should be understood as “only the processor can cause the ophthalmic laser treatment apparatus 1 to perform all the functions”. Moreover, the phrase “at least one of a circuit and a processor is configured to cause the ophthalmic laser treatment apparatus 1 to perform functions” should be understood as “the circuit can cause the ophthalmic laser treatment apparatus 1 to perform at least one of the functions and the processor can cause the ophthalmic laser treatment apparatus 1 to perform the remaining functions”. In the last example, if the ophthalmic laser treatment apparatus 1 performs functions A to C, for example, the functions A and B among the functions A to C may be implemented via a circuit, while the remaining function C may be implemented via a processor.”

### Trabecular Meshwork Treatment Mode

With reference to FIGS. 4 and 5, an example of a trabecular meshwork treatment mode executed by the ophthalmic laser treatment apparatus 1 in the present embodiment will be described. In this embodiment, as an example, the trabecular meshwork, which is an annular (partially arcuate) tissue among the tissues of the patient's eye E, is treated as a treatment target site. For example, Selective Laser Trabeculoplasty (SLT) is a treatment method in which therapeutic laser light is irradiated onto the trabecular meshwork of the anterior chamber angle of the patient's eye E in order to increase the outflow of aqueous humor. In SLT, therapeutic laser light is irradiated multiple times over the entire circumference or a part of the annular trabecular meshwork.

As shown in FIG. 4, in trabecular meshwork treatment of the present embodiment, a contact lens 26 is mounted on the cornea C of the patient's eye E. As an example, a gonioscope for observing the anterior chamber angle A of the patient's eye E, such as Goldmann's three-mirror lens, may be used as the contact lens 26. The contact lens 26 is provided with reflective surfaces (reflecting mirrors) 27. The anterior chamber angle A is observed through the reflective surface 27. Therefore, as shown in FIG. 5, in this embodiment, the entire anterior chamber angle A (the entire circumference) is not observed at the same time, but a part of the anterior chamber angle A is observed in a sector shape. The range of the sector-shaped portion of the anterior chamber angle A observed through the reflective surface 27 falls within an angular range of less than 180 degrees with the center of the anterior chamber angle A as a reference. The reflective surface 27 of the contact lens 26 reflects the therapeutic laser light and the aiming light in a direction intersecting the optical axis extending from the objective lens 25 (see FIGS. 2 and 3) toward the patient's eye E, thereby irradiating the trabecular meshwork TM, which is the treatment target site, with the therapeutic laser light and the aiming light. That is, in the observation state illustrated in FIG. 5, the trabecular meshwork TM of the anterior chamber angle A is irradiated with the therapeutic laser light through the reflective surfaces 27, whereby trabecular meshwork treatment is executed.

The operator adjusts the rotation angle of the contact lens 26 (that is, the angle in the rotational direction about the axis of the contact lens 26). As a result, the operator can adjust the reflection direction of the therapeutic laser light by the reflective surfaces 27 of the contact lens 26, as viewed from the line-of-sight direction of the operator in the observation optical system 40. The operator also operates the joystick unit 5 to move the base unit 4. As a result, the aiming position of the therapeutic laser light and the aiming light with respect to tissue of the patient's eye E is adjusted to the treatment spot S. With the aiming position adjusted, the operator operates an operation button of the joystick 5 or a foot switch. As a result, an execution instruction for irradiating the therapeutic laser light is input, and the therapeutic laser light is irradiated to the spot S. In SLT, in order to make it difficult to cause thermal denaturation of tissue, the therapeutic laser light is irradiated at lower output (energy and irradiation time) than in Argon Laser Trabeculoplasty (ALT). Therefore, it is difficult for the operator to visually recognize a state change (for example, a treatment mark) of the treatment site before and after treatment. As a surgical technique of SLT, it is known to fix a spot size of the therapeutic laser light and the aiming light to a predetermined size (for example, about 400 μm), and to intermittently irradiate the therapeutic laser light so that a plurality of irradiation spots are adjacent to each other along the trabecular meshwork TM. FIG. 5 schematically shows a state of the technique in which the therapeutic laser light is intermittently irradiated so that a plurality of irradiation spots are adjacent to each other. In SLT, however, it is difficult to visually recognize a treatment mark. That is, an irradiated spot S to which the therapeutic laser light has been irradiated is not visually recognized by the operator as shown in FIG. 5.

### Contact Lens

With reference to FIGS. 6 and 7, an example of the contact lens 26 used in trabecular meshwork treatment of the present embodiment will be described. The contact lens 26 shown in FIGS. 6 and 7 has a plurality of reflective surfaces 27. As a specific example, the contact lens 26 of the present embodiment is a four-mirror lens having four reflective surfaces 27 as reflective surfaces.

In detail, the contact lens 26 shown in FIG. 6 includes a lens barrel 26A and an optical portion 26B. The lens barrel 26A is substantially cylindrical and is gripped by fingers of a user. The optical portion 26B is fixed to the lens barrel 26A. A tip end (lower side in FIG. 6) of the optical portion 26B serves as a contact portion 26D that comes into contact with the patient's eye E. A transparent window is formed in the contact portion 26D.

Inside the optical portion 26B, the reflective surfaces 27 are formed. The reflective surfaces 27 are arranged at intervals of 90 degrees around an axis AX1. FIG. 7 shows a cross-section of the optical portion 26B taken along line A-A′ in FIG. 6. The cross-section A-A′ includes the axis AX1 and passes through centers of two opposing reflective surfaces 27 among the four reflective surfaces. As shown in FIG. 7, each reflective surface 27 is inclined at an angle of about 62 degrees with respect to a plane orthogonal to the axis AX1. With the pupil center of the patient's eye E approximately aligned with the center of the transparent window 29, most of the entire circumference of the anterior chamber angle (at least more than half of the region) can be observed via the four reflective surfaces 27, and therapeutic laser light can be irradiated. Therefore, by further rotating the contact lens 26 by about 45 degrees, observation and irradiation of therapeutic laser light to the remaining portion can be performed.

In the present embodiment, irradiation of the therapeutic laser light onto the entire circumference of the trabecular meshwork TM is performed while maintaining a state in which the pupil center of the patient's eye E is approximately aligned with the center of the transparent window 29. In this case, in order to align the aiming position with a site observed through each of the four reflective surfaces 27, the joystick unit 5 is operated.

With reference to FIG. 8, an example of an irradiation plan of the therapeutic laser light in the present embodiment will be described. FIG. 8 is a diagram illustrating an outline of an irradiation plan in a case where the therapeutic laser light is irradiated using the contact lens 26 shown in FIGS. 6 and 7.

In the present embodiment, as the irradiation plan, an irradiation order of the therapeutic laser light for a plurality of irradiation sections is determined. In this embodiment, each of eight divisions of the entire circumference of the annular treatment target site (the trabecular meshwork TM in this embodiment) is referred to as sections. As shown in FIG. 8, the eight sections C1 to C8 are broadly classified into first sections (C1 to C4 in this embodiment) and second sections (C5 to C8 in this embodiment). The first sections C1 to C4 and the second sections C5 to C8 are arranged alternately one by one. The sections C1 to C4 correspond respectively to the four reflective surfaces of the contact lens 26 when rotation angles of the four reflective surfaces are disposed at positions of 0°, 90°, 180°, and 270°(i.e., the first angles) with respect to the axis AX1 (in other words, the contact lens is fixed at a first orientation). The sections C5 to C8 correspond respectively to the four reflective surfaces of the contact lens 26 when the rotation angles of the four reflective surfaces are disposed at positions of 45°, 135°, 225°, and 315°(i.e., the second angles) with respect to the axis AX1 (in other words, the contact lens is at a second orientation).

Up to about 12 to 13 irradiation spots may be arranged within one section. However, the number and spacing of irradiation spots in each section may be changed according to an operation instruction by the operator. For example, the control unit 60 may calculate the number of scheduled irradiation spots according to an interval between two adjacent spots input by the operator and parameters of the scheduled irradiation region (for example, a circumferential length of the treatment target site).

As one example, in the present embodiment, the scheduled irradiation region and the number of scheduled irradiation spots are set together in units of sections. The operator may be able to select any combination of the eight sections via the control panel or the like. For convenience, the section in which the scheduled irradiation region and the number of scheduled irradiation spots are set is referred to as an irradiation section.

In a case where multiple irradiation sections are selected, the irradiation order of the therapeutic laser light for the irradiation sections is determined according to the arrangement of the four reflective surfaces 27 of the contact lens 26. That is, after irradiating the therapeutic laser light to all the first sections among the plurality of irradiation sections, the irradiation order is determined such that the therapeutic laser light is irradiated to each of the second sections. It should be noted that irradiation of the therapeutic laser light multiple times in each irradiation section is sequentially executed in a clockwise direction or a counterclockwise direction.

As one specific example, the irradiation plan will be described in a case where the scheduled irradiation region and the number of scheduled irradiation spots are set over the entire circumference of the anterior chamber angle A. In this case, all the eight sections are set as the irradiation sections. In this example, the irradiation order is defined as the order of numbers illustrated in each section as shown in FIG. 8. That is, first, irradiation of the therapeutic laser light is scheduled sequentially for the four sections C1 to C4 categorizes as the first sections, and then irradiation of the therapeutic laser light is scheduled sequentially for the four sections C5 to C8 categorized as the second sections. While irradiating the therapeutic laser light to the four sections C1 to C4 categorized as the first sections, and while irradiating the therapeutic laser light to the four sections C5 to C8 categorized as the second sections, it is not necessary to change the orientation of the contact lens 26 with respect to the patient's eye E. After completing irradiation of the therapeutic laser light to all the four sections categorized as the first sections, by changing the orientation of the contact lens 26 (i.e., by rotating the contact lens 26) only once at the timing before starting irradiation of the therapeutic laser light to the second sections, the therapeutic laser light can be irradiated over the entire circumference of the anterior chamber angle A.

It should be noted that, in the above description, the irradiation order is defined such that irradiation to the first sections is performed earlier than the second sections. However, the irradiation order may be defined as performing irradiation to the second sections first. Also, the first section among the eight sections C1 to C8 to which irradiation of the therapeutic laser light is performed first may be predetermined or may be selectable by the operator. Further, the irradiation order among the four sections C1 to C4 categorized as the first sections may be predetermined or may be selectable by the operator. The same applies to the second sections.

It should be noted that, in the present embodiment, as the contact lens 26, a four-mirror lens having the four reflective surfaces was described. However, the present disclosure is not necessarily limited thereto. The number of reflective surfaces of the contact lens 26 may be any integer of two or more. In this case, according to the number of reflective surfaces of the contact lens 26, positions of the irradiation sections, the number of the irradiation sections, and the irradiation order of the therapeutic laser light for the irradiation sections in the irradiation plan may be determined.

With reference to FIG. 9, a method of adjusting an aiming position of an irradiation spot will be described. In the present embodiment, as methods of adjusting the aiming position of the irradiation spot for the therapeutic laser light, there are (i) a method of adjusting a positional relationship between a patient's eye E and the laser irradiation optical system 10 by operating the joystick unit 5 and (ii) a method of performing at least one of a rotation operation and a movement operation of the reflective surfaces 27 of the contact lens 26. FIG. 9 (A) shows a state in which the most recent irradiation of the therapeutic laser light has been completed. In the state of FIG. 9 (A), a position of an irradiation completed spot SS where irradiation of the therapeutic laser light has been completed and a position of the aiming light AI coincide with each other. The operator needs to adjust the position of the aiming light AI (that is, the aiming position of the irradiation spot) from the state shown in FIG. 9 (A) to a position of the next irradiation spot (in the example shown in FIG. 9, a position to the right of the irradiation completed spot SS).

FIG. 9 (B) shows a state in which the aiming position has been adjusted to a position of the next irradiation spot by operating the joystick unit 5 (that is, in a state where the angles of the reflective surfaces 27 of the contact lens 26 are fixed) from the state shown in FIG. 9 (A). In FIG. 9 (B), the irradiation completed spot SS at the time of completion of the immediately preceding irradiation of the therapeutic laser light, and a position of the treatment target site (the trabecular meshwork TM in the present embodiment), are schematically shown by dotted lines. However, in practice, since no treatment mark is present on the irradiation completed spot SS at the time of completion of the immediately preceding irradiation, it is difficult to accurately adjust the aiming position by operating the joystick unit 5.

Further, FIG. 9 (C) shows a state in which the aiming position has been adjusted to a position of the next irradiation spot by rotating the reflective surfaces 27 of the contact lens 26 (that is, without operating the joystick unit 5) from the state shown in FIG. 9 (A). In FIG. 9 (C), the irradiation completed spot SS at the time of completion of the immediately preceding irradiation of the therapeutic laser light and a position of the treatment target site are schematically shown by dotted lines. However, in practice, also in FIG. 9 (C), since no treatment mark is present on the irradiation completed spot SS at the time of completion of the immediately preceding irradiation, it is difficult to accurately adjust the aiming position by rotating the reflective surfaces 27. In the present disclosure, adjustment of the aiming position by the operator is assisted by causing the internal display unit 50 to display a guide.

With reference to FIGS. 10 to 13, treatment control processing executed by the ophthalmic laser treatment apparatus 1 according to the present embodiment will be described. In the present embodiment, an example is illustrated in a case where treatment is performed using the contact lens 26 having a plurality of reflective surfaces 27 shown in FIGS. 6 and 7. In the present embodiment, the adjustment pattern of the aiming position of the therapeutic laser light executed is mainly a movement adjustment pattern in which a relative position of the ophthalmic laser treatment apparatus 1 with respect to the patient's eye is moved each time the aiming position is adjusted to each irradiation spot included in one irradiation section. However, in the movement adjustment pattern, in addition to moving the relative position of the ophthalmic laser treatment apparatus 1 with respect to the patient's eye, the contact lens may also be rotated. In the present embodiment, the entire circumference of the trabecular meshwork is divided into a plurality of sections (eight sections in this embodiment).

Then, the irradiation sections are defined as sections among the divided sections to which the therapeutic laser light is scheduled to be irradiated a prescribed number M (M≥2) of times (that is, irradiation of the therapeutic laser light for each of a prescribed number of irradiation spots is scheduled) by the irradiation plan. In the description of the present embodiment, the center O is assumed to be the point where the center of the display region of the internal display unit 50, the observation optical axis of the observation optical system 40, the optical axis of the therapeutic laser light, and the optical axis of the aiming light all coincide. In other words, the operator visually recognizes the spot AI of the aiming light at the center of the observation field and at the center of the display region of the internal display unit 50. The therapeutic laser light is irradiated onto the same spot as the spot AI of the aiming light.

First, with reference to FIG. 10, an example of an observation field of view of the operator during treatment by the ophthalmic laser treatment apparatus 1 of the present embodiment will be described. The ophthalmic laser treatment apparatus 1 causes the internal display unit 50 to display a spot interval guide 90 so that the operator can grasp intervals between the irradiation spots. The spot interval guide 90 indicates to the operator an appropriate interval between the irradiation spots to be irradiated with the therapeutic laser light.

As shown in FIG. 10, the ophthalmic laser treatment apparatus 1 in the present embodiment causes each interval between a plurality of indices included in the spot interval guide 90 to coincide with the appropriate interval the irradiation spots. Accordingly, when moving the aiming position of the therapeutic laser light from a previous irradiation spot to a next irradiation spot, the operator can make each interval between the irradiation spots closer to the appropriate interval by making the movement distance coincide with the interval between the indices in the spot interval guide 90. Thus, treatment according to the irradiation plan can be performed more appropriately.

The ophthalmic laser treatment apparatus 1 in the present embodiment causes the spot interval guide 90 to be displayed at a position separated from the aiming position of the therapeutic laser light (in FIG. 10, a position where the aiming light AI is projected) in the display region of the internal display unit 50 (in FIG. 10, at a position slightly above). In the example shown in FIG. 10, the spot interval guide 90 is displayed on the side opposite to the iris with respect to the treatment target site (the trabecular meshwork in this embodiment). However, the spot interval guide 90 may also be displayed on the iris side relative to the treatment target site.

The ophthalmic laser treatment apparatus 1 causes a next aiming index 90A for aligning a position of the next irradiation spot to be displayed at a position corresponding to the optical axis of the therapeutic laser light among the spot interval guide 90. Each time irradiation of the therapeutic laser light is executed, the ophthalmic laser treatment apparatus 1 moves the indices in the spot interval guide 90 by one appropriate interval of the irradiation spots in a direction opposite to a progress direction defined by the irradiation plan. Further, the ophthalmic laser treatment apparatus 1 moves the position of the next aiming index 90A in the spot interval guide 90 by one appropriate interval of irradiation spots in the progress direction defined by the irradiation plan. As a result, the position of the next aiming index 90A is maintained at a position corresponding to the aiming position. The operator can easily set the next aiming position by setting the position on the tissue where the next therapeutic laser light is to be irradiated, considering the position of the next aiming index 90A on the optical axis of the therapeutic laser light (and also on the optical axis of the aiming light when the aiming light is irradiated).

Among the indices in the spot interval guide 90, indices other than the next aiming index 90A are irradiation-completed indices 90B corresponding to spots where irradiation of the therapeutic laser light has already been completed, and unirradiated indices 90C corresponding to spots where the therapeutic laser light will be irradiated after the next irradiation. The ophthalmic laser treatment apparatus 1 causes the next aiming index 90A, the irradiation-completed indices 90B, and the unirradiated indices 90C to be displayed in different manner (that is, in manners that can be distinguished by the operator). Accordingly, the operator can easily grasp positional relationships among the next aiming index 90A, the irradiation-completed indices 90B, and the unirradiated indices 90C. As a result, treatment can be advanced more smoothly.

Each time irradiation of the therapeutic laser light is executed, the ophthalmic laser treatment apparatus 1 moves the entire spot interval guide 90 by one appropriate interval of the irradiation spots in a direction opposite to the progress direction defined by the irradiation plan. The operator adjusts the aiming position each time so that the indices that move each time irradiation of the therapeutic laser light is performed appear at the same position on the observation image observed through the observation optical system 40. As a result, adjustment of the aiming position can be easily performed. The operator can appropriately adjust each of the plurality of aiming positions by appropriately adjusting positional relationships between a characteristic portion existing in tissue included in the observation image and the positions of the indices of the spot interval guide 90. For example, the operator specifies one characteristic portion in the observation image. Then, the operator may execute a movement instruction to the apparatus so that, each time the spot interval guide 90 is moved by one appropriate interval of the irradiation spots (that is, each time irradiation of the therapeutic laser light is executed), one specific index (for example, the leftmost index or the second index from the left) among the plurality of indices coincides with the specified characteristic portion. That is, when the spot interval guide 90 includes the multiple indices, each of the aiming positions is appropriately adjusted by aligning at least one specific index among the indices with the characteristic portion.

The ophthalmic laser treatment apparatus 1 in the present embodiment displays some of the indices in the spot interval guide 90 to be arranged along a straight line. In the present embodiment, the ophthalmic laser treatment apparatus 1 displays the indices 90A, 90B, and 90C in the spot interval guide 90 to be arranged along a virtual straight angle reference line AL (which is not actually displayed on the internal display unit 50). In the present embodiment, centroids of the plurality of indices are arranged along the angle reference line AL. Each time irradiation of the therapeutic laser light is executed, the ophthalmic laser treatment apparatus 1 moves the spot interval guide 90 along the straight-line direction in which the indices are arranged. In this case, the operator can appropriately proceed with treatment by aligning the direction in which the indices are arranged with the moving direction for moving the aiming position of the therapeutic laser light to the next irradiation spot.

Further, in the present embodiment, the spot interval guide 90 includes an index 90D. The index 90D indicates a direction to one of the reflective surfaces 27 of the contact lens 26 that corresponds to the spot interval guide 90 being displayed. In the present embodiment, the index 90D is disposed on one side of the virtual straight angle reference line AL and apart from the line AL. As one example, in the present embodiment, the index 90D is displayed in the vicinity of the indices (indices 90A, 90B, and 90C) in the spot interval guide 90 that are linearly arranged (in FIG. 10, the index 90D is at a position slightly upward apart). A side of the spot interval guide 90 where the index 90D is disposed indicates the reflective surface 27 corresponding to the spot interval guide 90 currently being displayed. The operator can grasp the relative position between the patient's eye E and the laser irradiation optical system 10 based on the index 90D and, for example, the curvature direction of the trabecular meshwork TM. The operator can also adjust the relative position to a position corresponding to the appropriate reflective surface 27 as needed. In particular, in the present embodiment, the aiming light AI and the spot interval guide 90 are shown substantially at the center of the operator's observation field. Therefore, based only on the position of the spot interval guide 90, it is difficult to determine which reflective surface 27 corresponds to the spot interval guide 90 being displayed. In contrast, by the index 90D, the reflective surface 27 corresponding to the spot interval guide 90 being displayed is indicated, which is useful.

The ophthalmic laser treatment apparatus 1 in the present embodiment displays a peripheral guide 75 on the internal display unit 50 as the aiming guide. The peripheral guide 75 includes a next aiming guide 75A, an irradiation-completed guide 75B, and a non-irradiated guide 75C. The peripheral guide 75 indicates at least one of the rotation angle of the reflective surface of the contact lens appropriate to the progress in the irradiation plan (that is, an orientation of the reflective surface 27 with respect to the central axis AX1 of the contact lens 26), and a direction in which an irradiation spot to be irradiated with the therapeutic laser light is located (a direction in which the irradiation spot is located in the observation image using the contact lens 26). The peripheral guide 75 is displayed along the outer circumference of the observation field of view by the operator through the observation optical system 40. As described above, the observation optical axis by the observation optical system 40 coincides with the center of the display region of the internal display unit 50. Therefore, based on the center of the display region of the internal display unit 50, the peripheral guide 75 is displayed along the periphery of the observation field of view. Accordingly, the operator can grasp the appropriate direction.

In detail, in the present embodiment, a plurality of peripheral guides 75 are arranged to have an arc shape or annular shape along the periphery of the observation field of view. Further, when moving one peripheral guide 75, the control unit 60 moves it along an arc-shaped or annular line along the periphery of the observation field of view. The center of the arc-shaped or annular line along which the peripheral guides 75 are arranged (in other words, the center of the peripheral guides 75 or the center of curvature of the peripheral guide 75) coincides with the observation optical axis of the observation optical system 40. Therefore, the operator can appropriately grasp the direction indicated by the peripheral guide 75.

The peripheral guides 75 includes the next aiming guide 75A. The next aiming guide 75A indicates an appropriate direction of the irradiation spot to which the therapeutic laser light is to be irradiated next among a plurality of irradiation spots defined in the irradiation plan. Each time irradiation of the therapeutic laser light is executed, the control unit 60 moves the position of the next aiming guide 75A to a position corresponding to an adjacent irradiation spot in the progress direction defined in the irradiation plan. For example, in a case where irradiation of 100 shots is performed clockwise over the entire circumference of the trabecular meshwork TM in the irradiation plan, the next aiming guide 75A may be shifted by 3.6 degrees with the center O as a reference. Accordingly, the operator can appropriately grasp the direction of the irradiation spot to which the aiming position is to be adjusted next, by the next aiming guide moved along the periphery of the observation field of view.

The peripheral guides 75 includes the irradiation-completed guide 75B. The irradiation-completed guide 75B indicates the direction of an irradiation spot among a plurality of irradiation spots defined in the irradiation plan, where irradiation of the therapeutic laser light has already been completed. Each time irradiation of the therapeutic laser light is completed, the control unit 60 changes the next aiming guide 75A, which had been displayed immediately before irradiation, to the irradiation-completed guide 75B. Accordingly, the operator can grasp the direction in which irradiation of the therapeutic laser light has been completed and can appropriately adjust the next aiming position using the next aiming guide 75A. It also becomes easier to grasp the progress status of treatment.

The peripheral guides 75 include the non-irradiated guide 75C. The non-irradiated guide 75C indicates the direction of an irradiation spot among a plurality of irradiation spots defined in the irradiation plan, where irradiation of the therapeutic laser light is scheduled to be performed after the next irradiation time. Each time irradiation of the therapeutic laser light is completed, the control unit 60 changes the non-irradiated guide 75C, which was displayed in the direction of the irradiation spot of the next irradiation order defined in the irradiation plan, to the next aiming guide 75A. As a result, the next aiming guide 75A is moved. In this case, the operator can appropriately adjust the next aiming position using the next aiming guide 75A while also grasping the direction of irradiation spots to be irradiated after the next irradiation. Further, the operator can easily grasp the progress status of treatment.

The control unit 60 displays the next aiming guide 75A, the irradiation-completed guide 75B, and the non-irradiated guide 75C distinguishable manners (that is, in different display modes). Accordingly, the operator can easily recognize the type of the peripheral guides 75 being displayed. The control unit 60 may also selectively display one or two of the next aiming guide 75A, the irradiation-completed guide 75B, and the non-irradiated guide 75C. In this case, it is desirable that at least the next aiming guide 75A be displayed. It is more desirable that both the next aiming guide 75A and the irradiation-completed guide 75B be displayed. The next aiming guide 75A may be, for example, a straight line extending to the center O through which the optical axis of the observation optical system 40 passes. It is sufficient if the next aiming guide 75A can guide the direction in which the reflective surfaces 27 of the contact lens 26 are to be oriented.

The ophthalmic laser treatment apparatus 1 in the present embodiment displays the total number of irradiation spots defined in the irradiation plan. In the example shown in FIG. 10, the denominator of “SHOTS” represents the total number of irradiation spots. The number of irradiation spots where treatment has been completed (that is, the total number of irradiation spots where irradiation of the therapeutic laser light has been completed and irradiation spots skipped) is also displayed. In the example shown in FIG. 10, the numerator of “SHOTS” represents the number of irradiation spots where treatment has been completed. Each time irradiation of the therapeutic laser light or skipping of irradiation is executed, “1” is added to the numerator of “SHOTS. ” In the example shown in FIG. 10, a mode of the aiming light AI being used and the energy of the therapeutic laser light are also displayed.

With reference to the examples shown in FIGS. 11 and 12, a treatment flow using the spot interval guide 90 will be described. In FIGS. 11 and 12, parts other than the vicinity of the spot interval guide 90 displayed on the internal display unit 50 in the observation field of view of the operator are omitted, so that the state of transition of the spot interval guide 90 can be easily understood. FIGS. 11 and 12 illustrate cases where irradiation is performed with the actual region of the trabecular meshwork corresponding at the lower side (FIG. 11) corresponding to the irradiation section C1, and then at the left side (FIG. 12) corresponding to the irradiation section C2, based on the irradiation plan preset by the operator. In FIGS. 11 and 12, the lower trabecular meshwork appears in the observation field of view of the operator due to reflection by the reflective surface of the contact lens. Accordingly, in FIGS. 11 and 12, the image is inverted vertically, and the lower trabecular meshwork appears therein. On the reflective surface in FIGS. 11 and 12, when treatment for the irradiation spots proceeds from right to left (that is, counterclockwise), treatment for the irradiation spots proceeds from right to left (that is, clockwise) on the actual lower trabecular meshwork.

First, as shown in FIG. 11(a), the control unit 60 determines, according to progress of the irradiation plan, an angle of the spot interval guide 90 to be displayed on the internal display unit 50, and displays the spot interval guide 90 at the determined angle. The spot interval guide 90 shown in FIG. 11 is a guide for performing irradiation a prescribed number of ten times (that is, up to ten times) to the lower region of the actual trabecular meshwork TM. In the spot interval guide 90 shown in FIG. 11(a), the indices are arranged in the horizontal direction in order to perform treatment of the lower region of the trabecular meshwork TM. The control unit 60 identifiably displays, within the spot interval guide 90, the next aiming index 90A for aligning the aiming position for the next therapeutic laser light. In the example shown in FIG. 11(a), the control unit 60 first displays the next aiming index 90A at a position of an index located at an end on a side opposite to the progress direction defined in the irradiation plan (in FIG. 11(a), the index located at the right-hand end). Among the indices of the spot interval guide 90, the indices other than the one at a position overlapping the next aiming index 90A are rendered as the unirradiated indices corresponding, at least in part, to spots where the therapeutic laser light will be irradiated later than the next irradiation. Further, the control unit 60 displays the indices of the spot interval guide 90 so that the next aiming index 90A corresponds to a position on the optical axis of the therapeutic laser light irradiated by the laser irradiation optical system 10. As described above, a position where the next aiming index 90A is displayed need not coincide completely with the optical axis of the therapeutic laser light. That is, it suffices that the next aiming index 90A be displayed at a position corresponding to the aiming position of the therapeutic laser light so that the operator can recognize the aiming position to be irradiated with the therapeutic laser light.

The operator adjusts at least one of the rotation angle of the contact lens and the relative position between the patient's eye E and the laser irradiation optical system 10, which is changed by the joystick unit 5, such that a position of the next aiming index 90A in the spot interval guide 90 corresponds to a first irradiation spot of the therapeutic laser light in the treatment site of the patient's eye E (the trabecular meshwork in the present embodiment). Here, in a case where a characteristic portion is present in tissue of the patient's eye E appearing in the observation image, it is desirable that the operator grasp a positional relationship between at least one specific index among the indices of the spot interval guide 90 and the characteristic portion of the tissue. In this case, in subsequent treatment procedures, the operator adjusts the positional relationship between the specific index and the characteristic portion of the tissue to the same positional relationship. As a result, treatment for the irradiation spots can proceed smoothly. As shown in FIG. 11(b), the operator inputs an execution instruction for irradiating the therapeutic laser light in a state where the position of the next aiming index 90A is aligned with a position corresponding to the first irradiation spot of the therapeutic laser light in the treatment site of the patient's eye E (the trabecular meshwork in this embodiment). As a result, the therapeutic laser light is irradiated onto the spot S through which an optical axis of the laser irradiation optical system 10 passes.

As shown in FIG. 11(c), when irradiation of the therapeutic laser light is completed, the control unit 60 moves all the indices of the spot interval guide 90 by one appropriate interval between the irradiation spots. At this time, the control unit 60 moves all the indices of the spot interval guide 90 in a direction (in FIG. 11, the right direction) opposite to the progress direction (in FIG. 11, the left direction which is a clockwise direction) defined in the irradiation plan. The control unit 60 also moves the position of the next aiming index 90A by one appropriate interval of the irradiation spots in the progress direction defined by the irradiation plan. As a result, the position of the next aiming index 90A is fixed at a position corresponding to the aiming position. Further, the control unit 60 changes the index overlapped by the next aiming index 90A at the time of irradiation of the previous therapeutic laser light into the irradiation-completed index. As shown in FIG. 11(d), the operator mainly operates the joystick unit 5 to move tissue captured in the observation image, by the interval of the indices of the spot interval guide 90, in the treatment progress direction defined by the irradiation plan. Accordingly, the operator can adjust the aiming position of the next therapeutic laser light. The operator also adjusts a positional relationship between at least one specific index among the indices and the characteristic portion of the tissue to the same positional relationship as that in the previous irradiation. As a result, the aiming position of the next therapeutic laser light can also be adjusted, and the aiming position is appropriately adjusted. The operator aligns the position of the next aiming index 90A with a position corresponding to the aiming position of the next therapeutic laser light (the state shown in FIG. 11(d)). In this state, the operator inputs the execution instruction for irradiating the therapeutic laser light.

As shown in FIG. 11(e), when irradiation of the therapeutic laser light to the second irradiation spot is completed, the control unit 60 further moves all the indices of the spot interval guide 90 by one appropriate interval of the irradiation spots in the direction opposite to the progress direction defined by the irradiation plan. The control unit 60 also moves the position of the next aiming index 90A by one appropriate interval of the irradiation spots in the progress direction defined by the irradiation plan. As a result, the position of the next aiming index 90A is fixed at a position corresponding to the aiming position. The operator inputs an execution instruction for irradiating the therapeutic laser light in a state where the position of the next aiming index 90A is aligned with a position corresponding to the next irradiation spot of the therapeutic laser light (the state shown in FIG. 11(f)). The above procedure is repeated until irradiation of the therapeutic laser light is completed for all the irradiation spots in one irradiation section (in the example shown in FIGS. 11 and 12, twelve spots). As described above, the irradiation section refers to an angular range of an arc-shaped region in which irradiation of the therapeutic laser light a predetermined number M (M≥2 (M=12 in the present embodiment)) of times is scheduled to be executed based on the spot interval guide 90.

When irradiation of the therapeutic laser light the predetermined number M of times to one irradiation section (in the examples shown in FIGS. 11 and 12, twelve times of irradiation of the therapeutic laser light) is completed, the state becomes as shown in FIG. 12(a). The control unit 60 automatically entirely rotates the indices of the spot interval guide 90 in the progress direction by an angle corresponding to one irradiation section. The control unit 60 also displays the next aiming index 90A at a position overlapping with an index located at an end opposite to the treatment progress direction defined by the irradiation plan, among the indices. Further, the control unit 60 sets all the indices other than the one at a position overlapping with the next aiming index 90A among the indices of the spot interval guide 90 as the unirradiated indices. The control unit 60 displays the indices of the spot interval guide 90 and the next aiming index 90A so that the next aiming index 90A corresponds to a position on the optical axis of the therapeutic laser light irradiated by the laser irradiation optical system 10. As a result, the observation image becomes as shown in FIG. 12(b). The operator rotates the reflective surfaces of the contact lens clockwise, which is the treatment progress direction (see FIG. 12(c)). As described above, thereafter, irradiation of the therapeutic laser light is repeated for all the irradiation spots in the new irradiation section.

The ophthalmic laser treatment apparatus 1 in the present embodiment can also accept an instruction from the user to entirely rotate the spot interval guide 90, even before irradiation of the therapeutic laser light to all the irradiation spots within the currently treated irradiation section is completed (that is, in the middle of treatment for the irradiation spots within the irradiation section). When the instruction to rotate the spot interval guide 90 is input before treatment for all the irradiation spots in the irradiation section is completed, the ophthalmic laser treatment apparatus 1 entirely rotates the spot interval guide 90 by an angle corresponding to the range in which treatment has been performed within the irradiation section under treatment. Accordingly, even if treatment within each irradiation section is not completed, the operator can rotate the spot interval guide 90 and then proceed to treatment for the next irradiation section.

The ophthalmic laser treatment apparatus 1 in the present embodiment can also accept an instruction from the user (hereinafter referred to as a “skip instruction”) to skip irradiation of the therapeutic laser light to a next irradiation spot defined by the irradiation plan. When the skip instruction is input, the ophthalmic laser treatment apparatus 1 moves the spot interval guide 90, without irradiating the therapeutic laser light, by one appropriate interval of the irradiation spots in either the progress direction defined by the irradiation plan or the direction opposite to the progress direction. Accordingly, when a non-irradiated portion exists where irradiation of the therapeutic laser light is inappropriate, the operator can input the skip instruction to skip irradiation of the therapeutic laser light to the non-irradiated portion. Thereafter, the operator can also resume treatment from the irradiation spot that is scheduled subsequently. Thus, treatment can be smoothly advanced. That is, the ophthalmic laser treatment apparatus 1 is provided with a skip means for skipping irradiation of the therapeutic laser light to part of the irradiation spots defined by the treatment plan. Accordingly, both convenience provided by the treatment plan (for example, guidance of irradiation of the therapeutic laser light) and flexible treatment provided by the skip means (for example, a procedure for omitting irradiation of the therapeutic laser light to a non-irradiated portion found after starting treatment according to the treatment plan) can be realized.

As described above, also in the present embodiment, the spot interval guide 90 for allowing the user to grasp the appropriate intervals of the irradiation spots is displayed on the internal display unit 50 in accordance with progress of the irradiation plan. Accordingly, the operator can check the spot interval guide 90 while observing the patient's eye E through the eyepiece lens 46 (that is, without taking the eye away from the eyepiece lens 46). Then, the operator can adjust the aiming positions of the therapeutic laser light with reference to the confirmed spot interval guide 90.

The control unit 60 changes the intervals between the indices of the spot interval guide 90 displayed on the internal display unit 50 according to a magnification of the observation optical system 40. Accordingly, even when the observation magnification is changed, the spot interval guide 90 having appropriate intervals of the irradiation spots is displayed on the internal display unit 50.

With reference to FIG. 13, treatment control process in the present embodiment will be described. First, the control unit 60 acquires an irradiation plan of the therapeutic laser light for the patient's eye E (S81). In the treatment plan acquired in S81, an angle between two adjacent irradiation spots, an angle for one irradiation section, the number M of times the therapeutic laser light is irradiated within each irradiation section, the total number N of times the therapeutic laser light is irradiated over the entirety of the treatment target region, the irradiation spot at which the therapeutic laser light is first irradiated, and the irradiation order (including the irradiation direction) of the therapeutic laser light are defined.

The control unit 60 displays the spot interval guide 90 on the internal display unit 50 at an angle corresponding to the arrangement (orientation) of the first irradiation section (S82). The control unit 60 sets a value of a total irradiation number counter “n,” which specifies a cumulative number of times the therapeutic laser light has been irradiated over the entire treatment target region, to “0” (S83). Further, the control unit 60 sets a value of an inter-section irradiation number counter “m,” which specifies the number of times the therapeutic laser light has been irradiated within one irradiation section, to “0” (S84). The control unit 60 sets a display position of the next aiming index 90A, which serves as an index for irradiating the therapeutic laser light next, to a position overlapping with an index located at an end opposite to the progress direction of the irradiation order, among the plurality of indices of the spot interval guide 90 (S85).

The control unit 60 determines whether the execution instruction for irradiating the therapeutic laser light has been input by the operator (S87). If the execution instruction has not been input (S87: NO), the control unit 60 determines whether the skip instruction, which is an instruction to omit irradiation of the therapeutic laser light to the next irradiation spot defined by the irradiation plan, has been input (S88). If the skip instruction has not been input (S88: NO), the control unit 60 determines whether the intermediate rotation instruction, which is an instruction to rotate the entire spot interval guide 90 before irradiation of the therapeutic laser light is completed for all the irradiation spots in the currently treated irradiation section, has been input (S89). If none of the instructions in S87-S89 has been input, the determinations in S87-S89 are repeated, resulting in a standby state. During this time, the aiming position of the therapeutic laser light is adjusted by the operator.

When the execution instruction for irradiating the therapeutic laser light is input (S87: YES), the therapeutic laser light is irradiated (S90). The control unit 60 adds “1” to each of the values of the total irradiation number counter “n” and the inter-section irradiation number counter “m” (S91). The control unit 60 determines whether the value of the total irradiation number counter “n” has reached the total number “N” of times the therapeutic laser light is irradiated to the entire treatment target region (S92). If irradiation of the therapeutic laser light to all the irradiation spots has not been completed (that is, if “n” has not reached “N”) (S92: NO), the control unit 60 determines whether irradiation of the therapeutic laser light a predetermined number of times within the currently treated irradiation section has been completed (S93). That is, the control unit 60 determines whether the value of the inter-section irradiation number counter “m” has reached the irradiation number “M” of the therapeutic laser light within one irradiation section. If irradiation of the therapeutic laser light within the irradiation section under treatment has not been completed (S93: NO), the control unit 60 moves (shits) the entire spot interval guide 90 by one appropriate interval between the irradiation spots in the direction opposite to the progress direction of the irradiation spots (S94). In S94, the control for moving the next aiming index 90A by one appropriate interval is also executed. Thereafter, the process returns to S87.

When irradiation of the therapeutic laser light within one irradiation section has been completed (S93: YES), the control unit 60 rotates the spot interval guide 90 by an angle (the predetermined angle) corresponding to one irradiation section (S95). The control unit 60 resets the next aiming index 90A and the unirradiated indices (S98), and the process returns to S84. When irradiation of the therapeutic laser light to all the irradiation spots has been completed (S92: YES), the process is terminated.

Further, when the skip instruction is input before the execution instruction for irradiating the therapeutic laser light is input (S88: YES), the control unit 60 adds “1” to each of the values of the total irradiation number counter “n” and the inter-section irradiation number counter “m” (S91), and executes the processes of S92-S95 and S98. That is, if irradiation of the therapeutic laser light to all the irradiation spots has not been completed (if “n” has not reached “N”) (S92: NO), the control unit 60 moves the spot interval guide 90 by one appropriate interval in either the progress direction of the irradiation spots or the direction opposite to the progress direction, without irradiating the therapeutic laser light (S94). The direction in which the spot interval guide 90 is moved may be defined according to the instruction input by the operator. If “n” has reached “N” (S92: YES), the control unit 60 rotates the spot interval guide 90 by the predetermined angle (S95), and resets the next aiming index 90A and the unirradiated indices (S98).

Further, when the intermediate rotation instruction is input before the execution instruction for irradiating the therapeutic laser light is input (S89: YES), the control unit 60 calculates an angle corresponding to the range in which treatment has proceeded within the irradiation section under treatment, as an angle by which the spot interval guide 90 is to be rotated (S96). A specific calculation method for the rotation angle in S96 can be appropriately selected. For example, when an angle between two adjacent irradiation spots, when viewed from a center of a virtual circle through which a plurality of planned irradiation spots pass, is defined as “A degrees”, and the number of irradiation spots for which treatment has performed within the irradiation section under treatment is defined as “m” (in the present embodiment, this corresponds to a value of the inter-section irradiation number counter m). In the present embodiment, the control unit 60 calculates the angle corresponding to the range in which treatment has performed within the irradiation section under treatment by “A degrees ×m.” The number “m” of the irradiation spots for which treatment has performed includes also a number of irradiation spots for which irradiation of the therapeutic laser light has been skipped by the skip instruction. The control unit 60 rotates the spot interval guide 90, by the angle calculated in S96, in the progress direction defined in the treatment plan (S97). In a case where the number “m” of the irradiation spots for which treatment has performed is “0,” the angle calculated in S96 becomes “0 degrees.” In this case, the spot interval guide 90 is not rotated in S97. Thereafter, a reset process for the next aiming index 90A and the unirradiated indices is executed (S98), and the process returns to S84.

As described above, embodiments of the technology disclosed herein have been explained in detail, but these are merely illustrative and do not limit the scope of the claims. The technology described in the claims includes various modifications and alterations of the embodiments exemplified above.

1. An ophthalmic laser treatment apparatus that irradiates therapeutic laser light to tissue of a patient's eye each time an execution instruction for irradiating the therapeutic laser light is input, the ophthalmic laser treatment apparatus comprising:
a laser irradiation optical system irradiating the therapeutic laser light to the patient's eye;
an observation optical system causing an operator to observe an observation image of the patient's eye through an eyepiece lens;
an internal display unit provided in the observation optical system and configured to display an image to the operator through the eyepiece lens; and
a control unit, wherein
a scheduled irradiation region is defined as a region including a plurality of irradiation spots to which the therapeutic laser light is scheduled to be irradiated while using a contact lens having a plurality of reflective surfaces that reflect the therapeutic laser light in a direction intersecting with an optical axis,
the scheduled irradiation region is divided into a plurality of irradiation sections, and
the control unit is configured to execute:
an irradiation plan acquisition step of acquiring an irradiation plan that defines, in accordance with an arrangement of the plurality of reflective surfaces, an irradiation order of the therapeutic laser light for the plurality of irradiation sections; and
an aiming guide display step of controlling, in accordance with progress of the irradiation plan, the internal display unit to display an aiming guide for assisting the operator in adjusting an aiming position of the therapeutic laser light to an appropriate position.

2. The ophthalmic laser treatment apparatus according to claim 1, wherein
the plurality of irradiation sections includes a plurality of first irradiation sections and a plurality of second irradiation sections,
each of the plurality of first irradiation sections corresponds to a respective one of the plurality of reflective surfaces when the contact lens is fixed with the plurality of reflective surfaces at first angles,
each of the plurality of second irradiation sections corresponds to a respective one of the plurality of reflective surfaces when the contact lens is fixed with the plurality of reflective surfaces at second angles that are different from the first angles, and
the control unit is configured to acquire the irradiation plan that defines the irradiation order of the therapeutic laser light for the plurality of irradiation sections such that after the therapeutic laser light is irradiated to all the plurality of first irradiation sections, the therapeutic laser light is irradiated to each of the plurality of second irradiation sections.

3. The ophthalmic laser treatment apparatus according to claim 2, wherein
when irradiation of the therapeutic laser light to all the plurality of first irradiation sections has been completed, the control unit is further configured to execute a rotation recommendation step of recommending the operator to rotate the plurality of reflective surfaces of the contact lens at the second angles in accordance with progress of the irradiation plan.

4. The ophthalmic laser treatment apparatus according to claim 1, wherein
a center of a display region in the internal display unit coincides with an optical axis of the observation optical system.

5. The ophthalmic laser treatment apparatus according to claim 1, wherein
the control unit is further configured to control the internal display unit to display, as the aiming guide, an angle guide indicating a rotation angle of the reflective surface of the contact lens in accordance with progress of the irradiation plan.

6. The ophthalmic laser treatment apparatus according to claim 5, wherein
the control unit is further configured to control the internal display unit to display, as a target site guide serving as a reference with which an arcuate or annular treatment target site of the patient's eye observed by the operator through the observation optical system is aligned, the angle guide indicating the rotation angle of the reflective surface of the contact lens in accordance with progress of the irradiation plan.

7. The ophthalmic laser treatment apparatus according to claim 1, wherein
the control unit is further configured to control the internal display unit to display, as the aiming guide, a peripheral guide along a peripheral portion of an observation field observed by the operator through the observation optical system, and
the peripheral guide indicates at least one of a rotation angle of the reflective surface of the contact lens in accordance with progress of the irradiation plan and a direction of the irradiation spot to which the therapeutic laser light is to be irradiated.

8. A method executed by a control unit of an ophthalmic laser treatment apparatus that irradiates therapeutic laser light to tissue of a patient's eye each time an execution instruction for irradiating the therapeutic laser light is input, the method comprising:
irradiating, with a laser irradiation optical system, the therapeutic laser light to the patient's eye;
causing, with an observation optical system, an operator to observe an observation image of the patient's eye through an eyepiece lens; and
displaying, with an internal display unit provided in the observation optical system, an image to the operator through the eyepiece lens, wherein
a scheduled irradiation region is defined as a region including a plurality of irradiation spots to which the therapeutic laser light is scheduled to be irradiated while using a contact lens having a plurality of reflective surfaces that reflect the therapeutic laser light in a direction intersecting with an optical axis,
the scheduled irradiation region is divided into a plurality of irradiation sections, and
the method further comprises, with the control unit:
acquiring an irradiation plan that defines, in accordance with an arrangement of the plurality of reflective surfaces, an irradiation order of the therapeutic laser light for the plurality of irradiation sections; and
controlling, in accordance with progress of the irradiation plan, the internal display unit to display an aiming guide for assisting the operator in adjusting an aiming position of the therapeutic laser light to an appropriate position.

9. The method according to claim 8, wherein
the plurality of irradiation sections includes a plurality of first irradiation sections and a plurality of second irradiation sections,
each of the plurality of first irradiation sections corresponds to a respective one of the plurality of reflective surfaces when the contact lens is fixed with the plurality of reflective surfaces at first angles,
each of the plurality of second irradiation sections corresponds to a respective one of the plurality of reflective surfaces when the contact lens is fixed with the plurality of reflective surfaces at second angles that are different from the first angles, and
the method further comprises acquiring, with the control unit, the irradiation plan that defines the irradiation order of the therapeutic laser light for the plurality of irradiation sections such that after the therapeutic laser light is irradiated to all the plurality of first irradiation sections, the therapeutic laser light is irradiated to each of the plurality of second irradiation sections.

10. A non-transitory, computer readable, storage medium storing computer program code executed by a control unit of an ophthalmic laser treatment apparatus that irradiates therapeutic laser light to tissue of a patient's eye each time an execution instruction for irradiating the therapeutic laser light is input, the computer program code, when executed by the control unit, causing the ophthalmic laser treatment apparatus to perform:
irradiating, with a laser irradiation optical system, the therapeutic laser light to the patient's eye;
causing, with an observation optical system, an operator to observe an observation image of the patient's eye through an eyepiece lens; and
displaying, with an internal display unit provided in the observation optical system, an image to the operator through the eyepiece lens, wherein
a scheduled irradiation region is defined as a region including a plurality of irradiation spots to which the therapeutic laser light is scheduled to be irradiated while using a contact lens having a plurality of reflective surfaces that reflect the therapeutic laser light in a direction intersecting with an optical axis,
the scheduled irradiation region is divided into a plurality of irradiation sections, and
the computer program code further causes the ophthalmic laser treatment apparatus to perform:
acquiring, with the control unit, an irradiation plan that defines, in accordance with an arrangement of the plurality of reflective surfaces, an irradiation order of the therapeutic laser light for the plurality of irradiation sections; and
controlling, with the control unit in accordance with progress of the irradiation plan, the internal display unit to display an aiming guide for assisting the operator in adjusting an aiming position of the therapeutic laser light to an appropriate position.

11. The non-transitory, computer readable, storage medium according to Claim 10, wherein
the plurality of irradiation sections includes a plurality of first irradiation sections and a plurality of second irradiation sections,
each of the plurality of first irradiation sections corresponds to a respective one of the plurality of reflective surfaces when the contact lens is fixed with the plurality of reflective surfaces at first angles,
each of the plurality of second irradiation sections corresponds to a respective one of the plurality of reflective surfaces when the contact lens is fixed with the plurality of reflective surfaces at second angles that are different from the first angles, and
the computer program code further causes the ophthalmic laser treatment apparatus to perform acquiring, with the control unit, the irradiation plan that defines the irradiation order of the therapeutic laser light for the plurality of irradiation sections such that after the therapeutic laser light is irradiated to all the plurality of first irradiation sections, the therapeutic laser light is irradiated to each of the plurality of second irradiation sections.
